(19) United States
(12) Patent Application Publication
(10) Pub. No.: US 2025/0324967 A1
(43) Pub. Date: Oct. 23, 2025

(54) WATER-SOLUBLE WET WIPES

(71) Applicant: Health and Earth Care Solutions (PTY) Ltd., Midrand (ZA)

(72) Inventors: Hendrik Gerhardus Du Plessis, Somerset West (ZA); Rachel Cornelia Rossouw, Stellenbosch (ZA)

(21) Appl. No.: 18/698,682

(22) PCT Filed: Apr. 12, 2023
(86) PCT No.: PCT/IB2023/053744

§ 371 Date: Apr. 4, 2024

(30) Foreign Application Priority Data
Apr. 13, 2022 (ZA) 2022/04168
Jul. 4, 2022 (GB) 2209817.2

(51) Int. Cl. A01N 25/34; A01N 31/06; A01N 65/22; A01P 17/00

(52) U.S. Cl. CPC A01N 25/34; A01N 31/06; A01N 65/22; A01P 17/00

(57) ABSTRACT

The invention describes a water-soluble wet wipe comprising (i) a substrate comprising, as the only polymer, a thermally bonded non-woven polyvinyl alcohol fibre of 94% (w/w) or more and (ii) a liquid infusion comprising glycerine and at least 5% or at least 10% water, and one or more alcohol(s) or metal salt(s), or both one or more alcohol(s) and metal salt(s), and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, protein(s) or peptide(s) including collagen and elastin, and surfactant(s). The invention further describes a method of manufacturing the water-soluble wet wipe, a liquid infusion for infusing the thermally bonded non-woven polyvinyl alcohol fibre substrate to form the water-soluble wet wipe and methods for use of the water-soluble wet wipe.

### FIELD OF THE INVENTION

The current invention relates to water-soluble wet wipes. The water-soluble wet wipes may in particular be infused with a composition suitable for any one or more of refreshing, sanitizing or disinfecting, repelling insects, removing nail-polish or removing make-up.

### BACKGROUND OF THE INVENTION

COVID-19 has changed the way the world views health and hygiene. Nonwoven materials, which wet wipes are made of, have seen an exponential growth in usage during the course of the pandemic. Its low cost has ensured that this material has seen an immense uptake in use as a carrier for disinfectant infusions, used for sanitizing and hygiene purposes.

Current market nonwovens take decades and even centuries to decompose in landfill sites. Furthermore, wipes marketed as ‘biodegradeable’ may boast decomposition periods of a year, or even less, but fail to mention that this only holds true under the right environmental conditions such as temperature and sunlight.

With the arrival of COVID-19, society has become increasingly more aware of public sanitation, disinfection, as well as hygienic practices. This has driven the exponential growth in the wet wipe market and will continue to do so well into the future.

Wet wipes are now of significant environmental concern and as they are bigger, heavier, thicker, denser, and much stronger than toilet paper, shouldn't be flushed down any drainage system as they don't break down the way toilet paper does. These wipes instead clog homeowner and municipal sewer pipes. They also put stress on wastewater collection and treatment equipment, and cause cities to spend thousands and even millions on premature equipment repair and replacement.

Wet wipe accumulation in drainage/sewer systems have led to the production of what are known as “fatbergs” blocking sewer systems. Wipes adhere to fats, oils and grease in the drainage system, creating “plugs” or “fatbergs” in residential and municipal pipelines. These fatbergs continue to grow and can render an entire pipeline inoperative. Fatbergs can even be drawn into municipal water treatment plants, clogging and damaging the pumps in the plant. Municipalities need to manually clear out pumps and remove clogs.

“Flushable wipes” are often similar to non-flushable wipes on the basis of their physical and mechanical attributes. ‘Flushable’ claims hence only hold true for the manufacturer and not for the consumer, unless the material has proven to be entirely water dissolvable.

Furthermore, wet wipes contribute to microplastic pollution in the world's rivers and oceans. Additives used in the manufacturing of wet wipes often contain toxins such as cadmium and lead, which is then released when the wipe decomposes over many years. Furthermore, many fish and other marine life are ingesting the nonwoven materials in the water and dying as a result. Wet wipe reefs and even islands are forming in rivers and in the ocean. Wet wipes are ending up in the natural vegetation, polluting the natural habitat of many animal species as the materials take many years to decompose-all the while releasing harmful toxins.

Bioaccumulation is a process of accumulation of chemicals in an organism that takes place if the rate of intake exceeds the rate of excretion. Chemicals are introduced into the organism through exposure to the abiotic environment (soil, water, air) or as dietary intake (trophic transfer). Current wet wipes, as they disintegrate have been found to release various toxins including nonwoven materials, and lead, that bioaccumulate.

There is therefore a need for a wet wipe that can disintegrate without causing blockages and that doesn't release toxins that would accumulate in the environment over time cadmium resulting in harm to plants, fish and animals.

Although water-soluble pads (rather than wet-wipes) of a polyvinyl alcohol substrate with a cleaning solution have been disclosed in U.S. Pat. No. 8,197,830B2, these have the disadvantage that the pads require coating with a cleaning solution that has a low water content, as the structural integrity of the substrate would be compromised should a solution that has a high water content be used. Therefore, the solution is surface-coated onto the substrate, and surface tension and surface texture function to retain the solution on the substrate. Furthermore, the pads are preferably made up of between 70 to 99.5% (w/w) of polyvinyl alcohol substrate, and from 30 to 0.5% (w/w) of the cleaning solution, so as to ensure that the integrity of the pads can be maintained. This limits the volume of cleaning solution that can be delivered, and therefore also the efficacy of the pads to perform a cleaning function. Water content must be preferably 2%, and, most preferably 1% weight/weight (w/w). This reduces the wetting and cleaning efficacy of the pads.

GB2018053218 discloses a water-soluble wet wipe comprised of a polyvinyl alcohol substrate, but no water may be present in the infused cleaning or moisturising solution or the integrity of the wet-wipe is compromised. This therefore limits the possible cleaning or moisturising formulations that may be used with the wet wipe. Furthermore, due to the use of various mineral and other oils for the protection of the integrity of the wet wipe, the wipe has an oily residue which is not desirable, particularly in the case of a refreshing wipe.

There is therefore a need for a water-soluble wet wipe that is infused with a solution that includes water but where the integrity of the infused wet-wipe is maintained during storage and whilst in use, until it is disposed of by flushing or another means of dissolution in water. It would be beneficial if the wipe had a soft and fluffy texture, and was pliable for effective and comfortable use on the body and face.

### SUMMARY OF THE INVENTION

According to a first aspect of the invention, there is provided a water-soluble wet wipe comprising or consisting of:
a substrate comprising, as the only polymer, a thermally bonded non-woven polyvinyl alcohol fibre of 94% weight/weight (w/w) of the total substrate content or more; and
a liquid infusion comprising or consisting of:
   glycerine and at least 5% or 10% (w/w of the total liquid infusion content) water, and
   one or more alcohol(s), or
   one or more metal salt(s), or
   both one or more alcohol(s) and one or more metal salt(s), and
   optionally one or more additives including chelating agent(s), stabiliser(s), emulsifier(s), preservative(s), exfoliating agent(s), hydrating agent(s), anti-oxidants, emollient(s), thickener(s), lubricant(s), acetone and/or ethyl acetate, essential oil(s), fragrance(s), hydrogen peroxide, one or more proteins or peptides such as collagen, and surfactant(s),
wherein where 0% alcohol(s) are included >0% metal salt(s) are present, and wherein where 0% metal salt(s) are included, >0% alcohol(s) are present.

There is no particular restriction as to the type of polyvinyl alcohol employed. Whilst it is typically the case that only a single polyvinyl alcohol is used, a blend of different polyvinyl alcohols may also be employed.

As one skilled in the art will appreciate, non-woven fibres can be fabricated via a range of different techniques such as electrospinning or melt-blowing. The invention is not limited to any particular type of non-woven fibre. Similarly, the process by which the non-woven fibres are thermally bonded is also not particularly and would be familiar for one skilled in the art. There is no particular restriction as to the dimensions of the fibres. The substrate may be formed from several individual layers of non-woven fibre sheets that are thermally bonded (or otherwise cohered) together. Alternatively, one to five layers of thermally bonded non-woven fibres material may be employed; more typically, one to three layers; even more typically one or two layers; and often a single layer of thermally bonded non-woven fibres may be employed material.

As used herein, the term ‘water-soluble’ is intended to encompass materials that dissolve at a particular rate when introduced into an abundance of water. The wipes of the invention are capable of carrying various aqueous, polar solutions whilst retaining their structural integrity for at least a defined period of time. However, when introduced to a large volume of water (i.e. typically after use and disposal) the wipes are able to breakdown into harmless soluble by-products. Typically, the wet wipes of the invention possess a solubility such that substantially all (at least 95% w/w) of the wet wipe will dissolve when left in an excess of stirred tap water at 30° C. in less than five minutes.

As used herein, the term ‘polymer’ is intended to mean any of a class of natural or synthetic substances composed of very large molecules (or macromolecules) that are multiples of monomeric chemical units.

The amount of polyvinyl alcohol used contained in the substrate may be in the range of 94% to 98% (w/w); more typically 94% to 96% (w/w) and even more typically 94% to 95% (w/w). In some embodiments, the amount of polyvinyl alcohol is equal to 94% (w/w) or more; often 96% (w/w) or more; and in some cases up to 98% (w/w) or more.

In particular, the substrate does not comprise any additional polymers other than polyvinyl alcohol. Patterns and/or images may also be fabricated onto the substrate of the wipes.

There is no particular limitation as to the choice of metal salts that can be employed, provided that said salts are substantially non-toxic. Typically, the choice of metal salt will be not promote skin dryness and will be relatively cheap and available. Metal salts can provide a variety of functionality, including: controlling the solubilising properties of the liquid infusion, balancing the pH of the infusion and acting as a preservative. Typical examples of metal salts include, but are not limited to salts of: benzoates, citrates, gluconates and chlorides. Typical counter-ions include sodium and potassium (most typically potassium). Sodium chloride or potassium chloride are typical metal salts used in the invention. Further in particular the one or more metal salt(s) may be potassium chloride. It may be the case that two or more metal salts are used, each of which may perform different roles in the liquid infusion.

The choice of alcohol employed in the liquid infusion is not particularly limited and may be selected from glycols (such as ethylene glycol or propylene glycol), isopropyl alcohol, ethyl alcohol or other alcohol suitable for sanitising or disinfecting. Of these, isopropyl alcohol is most typically used.

The wet wipes of the invention are typically suitable for use on skin, including of the body and/or face, or on nails including finger and toe nails. In an alternative embodiment of the invention, the wet wipes are suitable for use on a hard surface requiring cleaning such as a lens, or screen, including for spectacles, a television or monitor, computer, a mobile phone, or a tablet and the like.

The ratio of liquid infusion to substrate may vary as the wet wipes of the invention are intended for various applications and the composition of the liquid infusion may change based on a desired application. However, typically, the ratio of substrate to infusion is in the range of 1:1 to 1:10; often 1:1.5 to 1:8.5; more typically 1:2 to 1:8, even more typically 1:2 to 1:6, more typically still 1:2.5 to 1:5 respectively (weight for weight). Typically, the wet wipes of the invention will comprise up to and including 90% (w/w) liquid infusion; more typically up to and including 80% (w/w) liquid infusion; even more typically up to and including 70% (w/w); even more typically still up to and including 60% (w/w); and often up to and including 50% (w/w).

The substrate, when loaded with liquid infusion, typically has a thickness of in the range of 100 μm to 500 μm, more typically 150 μm to 450 μm; even more typically, 200 μm to 400 μm; more typically still 250 μm to 350 μm, and even more typically still 275 μm to 325 μm, or any range therebetween.

Whilst there are no particular limitations on the choice of additives, provided that said additives are substantially non-toxic, common additives that can be included in the liquid infusion include one or more: moisturisers or hydrating agents, surfactants, antibacterial agents, antifungal agents, antiviral agents, essential oils, fragrances, hydrogen peroxide, insect repellents, chelating agents, stabilisers, emulsifiers, preservatives, exfoliating agents, anti-oxidants, emollients, thickeners, lubricants, acetone or ethyl acetate, one or more proteins or peptides such as collagen, elastin and the like, and combinations thereof. For example the essential oil(s) may be peppermint, menthol (Mentha arvensis), tea tree oil (Melaleuca alternifolia), lemon oil including Citrus limon oil, sweet almond oil (Prunus amagdalis dulcis), jojoba oil (Simmondsia chinensis), or any combination thereof, but it is to be appreciated that any one or more essential oil(s) as desired may be used. Regarding the essential oils, any one or more essential oil(s) having antimicrobial, including anti-viral and/or anti-bacterial properties known to those skilled in the art may typically be used. This provides both antimicrobial properties and cosmetic properties in a single additive.

According to one particular embodiment of the invention, the additives may include any ethyl acetate, acetone or a mixture thereof, and optionally any one or more of essential oils including tea tree oil and sweet almond oil, and one or more additional fragrance(s).

According to a further particular embodiment of the invention, the additives may include any one or more of chelating agent(s), stabiliser(s), emulsifier(s), preservative(s), exfoliating agent(s), hydrating agent(s), anti-oxidants, emollient(s), thickener(s), one or more proteins or peptides such as collagen, elastin and the like and/or lubricant(s).

The total amount of additives present in the liquid infusion is typically less than 40% (w/w), more typically less than 35% (w/w), even more typically less than 25% (w/w), more typically still less than 20% (w/w) and most typically less than 15% (w/w).

The liquid infusion described herein comprises or consists of a mixture of glycerine and at least 5% or 10% (w/w) water to which various additional ingredients may be added, depending upon the particular application in question. Whist the exact ratio of the ingredients that make up the liquid infusion may vary, various typical embodiments of the invention are described below by way of illustration.

In one particular embodiment of the invention, the water-soluble wet wipe is a refreshing wet wipe for application of a liquid infusion to the face or body as desired, and the liquid infusion comprises or consists of:
water: 10-85% (w/w); and
glycerine: 2-25% (w/w); and
alcohol(s): 0-25% (w/w); and/or
metal salt(s): 0-35% (w/w); and
one or more surfactant(s): 0.5-10% (w/w); and
fragrance: 0.3-5% (w/w); and
optionally henoxyethanol and/or ethylhexylglycerin: 0-1% (w/w); and
optionally one or more solubilizer(s): 0-10% (w/w) and;
optionally one or more emulsifier(s): 0-15% (w/w) and;
optionally one or more emollient(s): 0-10% (w/w),
   wherein where 0% alcohol(s) are included >0% metal salt(s) are present, and wherein where 0% metal salt(s) are included, >0% alcohol(s) are present.

Typically, in this embodiment, the amount of water in the liquid infusion is in the range 15%-80% (w/w); more typically in the range 20%-75% (w/w); even more typically in the range 25%-70% (w/w); and most typically in the range 30%-65% (w/w). Typically, in this embodiment, the amount of glycerine in the liquid infusion is in the range 3%-23% (w/w); more typically in the range 5%-23% (w/w); even more typically in the range 10%-23% (w/w); and most typically in the range 15%-20% (w/w). Typically, in this embodiment, the amount of alcohol in the liquid infusion is in the range 1%-23% (w/w); more typically in the range 1%-15% (w/w); even more typically in the range 1%-10% (w/w); and most typically in the range 1%-5% (w/w). Typically, in this embodiment, the amount of metal salt(s) in the liquid infusion is in the range 1%-30% (w/w); more typically in the range 5%-25% (w/w); even more typically in the range 10%-25% (w/w); and most typically in the range 10%-25% (w/w).

Regarding the optional additives, in this embodiment, the amount of surfactant in the liquid infusion is in the range 0.5%-9% (w/w), more typically in the range 0.5%-8% (w/w). Typically, in this embodiment, the amount of fragrance in the liquid infusion is in the range 0.3%-4% (w/w); and more typically in the range 0.5%-3.5% (w/w).

More specifically, the liquid infusion of the refreshing wet wipe may consist of:
water: 10-85% (w/w); and
glycerine: 2-25% (w/w); and
alcohol(s): 0-25% (w/w); and/or
metal salt(s): 0-35% (w/w); and
surfactant(s): 0.5-10% (w/w); and
fragrance: 0.1-5% (w/w); and
henoxyethanol and/or ethylhexylglycerin: 0.01-1% (w/w); and
one or more solubilizer(s): 0-10% (w/w) and;
one or more emulsifier(s): 0-15% (w/w) and;
one or more emollient(s): 0-10% (w/w),
   wherein where 0% alcohol(s) are included >0% metal salt(s) are present, and wherein where 0% metal salt(s) are included, >0% alcohol(s) are present.

This water-soluble wet wipe may be for application of the liquid infusion to the face or body of an infant or an adult. In the case of an infant, the percentage alcohol(s) will typically be limited to 0-10% (w/w).

In a second embodiment of the invention, the water-soluble wet wipe is a sanitizing or disinfecting wet wipe for application of a liquid infusion on the body, excluding sensitive areas, or on general surfaces to be sanitised or disinfected as desired, and the liquid infusion may comprise or consist of:
water: 5-25% (w/w), or 10-24% (w/w);
glycerine: 1-20% (w/w);
alcohol: 70-95% (w/w);
one or more essential oil(s): 0.05-5% (w/w);
optionally hydrogen peroxide: 0-7% (w/w); and
optionally additional fragrance(s): 0.05-3% (w/w).

Typically, in this embodiment, the amount of water in the liquid infusion is in the range 5%-20% (w/w); more typically in the range 10%-18% (w/w); and even more typically in the range 10%-15% (w/w). Typically, in this embodiment, the amount of glycerine in the liquid infusion is in the range 1%-18% (w/w); more typically in the range 2%-15% (w/w); even more typically in the range 3%-12% (w/w); and most typically in the range 3%-8% (w/w). Typically, in this embodiment, the amount of alcohol in the liquid infusion is in the range 78%-92% (w/w); and more typically in the range 80%-90% (w/w).

Regarding the optional additives, in this embodiment, the amount of essential oil(s) in the liquid infusion is in the range 0.05%-4% (w/w); more typically in the range 0.1%-3% (w/w); and even more typically in the range 0.1%-2% (w/w). Typically, in this embodiment, the amount of hydrogen peroxide in the liquid infusion is in the range 1%-6% (w/w); more typically in the range 2%-5% (w/w); and even more typically 3%-4% (w/w) hydrogen peroxide. Typically, in this embodiment, the amount of fragrance in the liquid infusion is in the range 0.05%-2.5% (w/w); and more typically in the range 0.15%-2% (w/w).

More specifically, the liquid infusion of the sanitizing or disinfecting wet wipe may consist of:
water: 10-15% (w/w);
glycerine: 3-8% (w/w);
alcohol: 78-92% (w/w);
one or more essential oil(s): 0.05-4% (w/w);
hydrogen peroxide: 1-6% (w/w); and
additional fragrance(s): 0.05-2.5% (w/w).

In a third embodiment of the invention, the water-soluble wet wipe is an insect repellent, and optionally also disinfectant or sanitizing wet wipe, for application of a liquid infusion on the body, particularly uncovered parts of the body that may be exposed to insect bites, and the liquid infusion comprises or consists of:
water: 5-25% (w/w), or 10-24% (w/w);
glycerine: 1-25% (w/w);
alcohol: 55-95% (w/w);
insect repellent(s): 0.5-25% (w/w); and
optionally surfactant(s): 0.5-10% (w/w).

Typically, in this embodiment, the amount of water in the liquid infusion is in the range 5%-23% (w/w); more typically in the range 5%-15% (w/w); and even more typically in the range 10%-12% (w/w). Typically, in this embodiment, the amount of glycerine in the liquid infusion is in the range 2%-20% (w/w); more typically in the range 2%-15% (w/w); even more typically in the range 2.5%-12% (w/w); and most typically in the range 3%-8% (w/w). Typically, in this embodiment, the amount of alcohol in the liquid infusion is in the range 60%-90% (w/w); more typically in the range 65%-85% (w/w); and even more typically 70%-80% (w/w). Typically, in this embodiment, the amount of insect repellent in the liquid infusion is in the range 0.5%-20% (w/w); more typically in the range 5%-15% (w/w); and even more typically in the range 8%-12% (w/w).

Regarding the optional additives, in this embodiment, the amount of surfactant in the liquid infusion is in the range 1%-6% (w/w); more typically in the range 2%-5% (w/w); and even more typically 3%-4% (w/w) surfactant.

Typically, the repellent is an essential oil. For example the essential oil(s) may be citronella and/or lemongrass, but it is to be appreciated that any one or more essential oil(s) known to have insect repellent properties known to those skilled in the art may be used. Any suitable surfactant(s) known to those skilled in the art may be used. For example, the insect may be any one or more of mosquitoes, fleas, ticks, flies and the like. In particular the insect may be a mosquito.

More specifically, the liquid infusion of the insect repellent wipe may consist of:
water: 10-12% (w/w);
glycerine: 3-8% (w/w);
alcohol: 70-80% (w/w);
insect repellent(s): 8-12% (w/w); and
optionally surfactant(s): 3-4% (w/w).

According to a fourth embodiment of the invention, the water-soluble wet wipe is a nail-polish remover wet wipe for application of a liquid infusion to the finger or toenails of the body, thereby to remove nail-polish therefrom, wherein the liquid infusion comprises or consists of:
water: 10-20% (w/w);
glycerine: 3-15% (w/w);
alcohol(s): 5-20% (w/w);
ethyl acetate: 0-85% (w/w) and/or acetone: 0-90% (w/w) or a mixture thereof;
optionally essential oil(s): 0.4-10% (w/w); and
optionally additional fragrance(s): 0.2-3% (w/w),
   wherein where 0% acetone is included >0% ethyl acetate is present, and wherein where 0% ethyl acetate is included, >0% acetone is present.

Typically, the alcohol is isopropyl alcohol and the essential oil(s) are a combination of at least sweet almond oil and jojoba oil, but it is to be appreciated that any one or more essential oil(s) known to have moisturising or hydrating properties known to those skilled in the art may be used.

More specifically, the liquid infusion of the nail-polish remover wet wipe may consist of:
water: 10-20% (w/w);
glycerine: 3-15% (w/w);
alcohol(s): 5-20% (w/w);
ethyl acetate: 0-85% (w/w) and/or acetone: 0-90% (w/w) or a mixture thereof;
one or more essential oil(s): 0.4-10% (w/w); and
optionally additional fragrance: 0.2-3% (w/w),
   wherein where 0% acetone is included >0% ethyl acetate is present, and wherein where 0% ethyl acetate is included, >0% acetone is present.

According to a fifth embodiment of the invention, the water-soluble wet wipe is a cosmetic wet wipe for application of a liquid infusion to the face, including to clean, moisturise or remove make-up therefrom, wherein the liquid infusion comprises or consists of:
water: 10-70% (w/w); and
glycerine: 2-20% (w/w); and
one or more alcohol(s) including isopropyl alcohol: 0-20% (w/w); or
one or more metal salts including potassium chloride or sodium chloride or a combination thereof: 0-30% (w/w); or
both one or more alcohol(s) and one or more metal salt(s); and
DL-Alpha tocopheryl acetate or Vitamin E acetate: 0.1-5% (w/w); and
one or more essential oil(s): 1.8-18% (w/w); and
one or more emulsifier(s) including hydrogenated castor oil and/or polysorbate 20:2-15% (w/w); and
one or more chelating agents including disodium ethylenediaminetetraacetic acid (EDTA): 0.1-0.5% (w/w); and
one or more emollient(s) including isopropyl myristate: 1-7.0% (w/w); and
one or more surfactant(s) including cocamidopropyl betaine: 0.5-5.0 (w/w); and
one or more antioxidants including caprylic/capric triglyceride: 1.0-6.0% (w/w); and
one or more stabilisers and/or preservatives including phenoxyethanol and ethylhexylglycerin or a combination thereof: 0.5-1.0% (w/w); and
optionally mineral oil including paraffinum liquidum: 0-12% (w/w); and
optionally an exfoliant including lactic acid: 0-7% (w/w); and
optionally a hydrating agent including sodium hyaluronate or hyaluronic acid: 0-4% (w/w),
optionally one or more peptide(s) or protein(s) including a hydrolysed peptide or protein such as collagen or elastin: 0-10% (w/w), more preferably 0-2.5% (w/w),
   wherein where 0% alcohol(s) are included >0% metal salt(s) are present, and wherein where 0% metal salt(s) are included, >0% alcohol(s) are present.

More specifically, the liquid infusion of the cosmetic wet wipe may consist of:
water: 10-70% (w/w); and
glycerine: 2-20% (w/w); and
one or more alcohol(s) including isopropyl alcohol: 0-20% (w/w); or
one or more metal salts including potassium chloride or sodium chloride or a combination thereof: 0-30% (w/w); or
both one or more alcohol(s) and one or more metal salt(s); and
DL-Alpha tocopheryl acetate or Vitamin E acetate: 0.1-5% (w/w); and
one or more essential oil(s): 1.8-18% (w/w); and
one or more emulsifier(s) including hydrogenated castor oil and/or polysorbate 20:2-15% (w/w); and
one or more chelating agents including disodium ethylenediaminetetraacetic acid (EDTA): 0.1-0.5% (w/w); and
one or more emollient(s) including isopropyl myristate: 1-7.0% (w/w); and
one or more surfactant(s) including cocamidopropyl betaine: 0.5-5.0 (w/w); and
one or more antioxidants including caprylic/capric triglyceride: 1.0-6.0% (w/w); and
one or more stabilisers and/or preservatives including phenoxyethanol and ethylhexylglycerin or a combination thereof: 0.5-1.0% (w/w); and
mineral oil including paraffinum liquidum: 0.01-12% (w/w); and
an exfoliant including lactic acid: 0.01-7% (w/w); and
a hydrating agent including sodium hyaluronate or hyaluronic acid: 0.01-4% (w/w),
one or more peptide(s) or protein(s) including a hydrolysed peptide or protein such as collagen or elastin: 0.01-10% (w/w), more preferably 0.01-2.5% (w/w),
   wherein where 0% alcohol(s) are included >0% metal salt(s) are present, and wherein where 0% metal salt(s) are included, >0% alcohol(s) are present.

In one possible embodiment of the invention, the cosmetic wet wipe is in the shape of a mask, sized to fit a person's face, such that it can be laid onto the surface of the face and pressed down by a user to allow the active infusion to penetrate the skin, followed by removal and disposal of the cosmetic wet wipe mask.

Typically, the alcohol is isopropyl alcohol and the essential oil(s) are a combination of at least tea tree oil: 0.1-2.0% (w/w), lemon oil including citrus limon oil: 0.2-3.0% (w/w), sweet almond oil: 1.0-6.0% (w/w) and jojoba oil: 0.5-7.0% (w/w), but it is to be appreciated that any one or more essential oil(s) known to have moisturising or hydrating properties known to those skilled in the art may be used.

According to a sixth embodiment of the invention, the water-soluble wet wipe is a wet wipe for application of a liquid infusion to sensitive skin, including for a baby's skin, including to clean or moisturise the skin, wherein the liquid infusion comprises or consists of:
30-90% (w/w) water, more preferably from about 50-80% or from about 60 to 80% or from about 70 to 80% (w/w);
2-35% (w/w) glycerine, more preferably from about 5-25%, or from about 5-20%;
5-33% (w/w) potassium chloride, more preferably from about 5-25%, or from about 8-15%;
0.2-25% (w/w) polysorbate 20 or any other suitable emulsifiers or surfactants known to those skilled in the art, more preferably from about 1 to 10%, or from about 1 to 5%; optionally 0-10% (w/w) coco caprylate caprate or any other suitable light emollient oil for skin care known to those skilled in the art;
optionally 0-20% (w/w) C12-C15 alkyl benzoate;
optionally 0-2% (w/w) fragrance; and
optionally 0-3% (w/w) essential oil, including tea tree oil or any other suitable essential oil known to those skilled in the art.

More specifically, the liquid infusion of the wet wipe for application of a liquid infusion to sensitive skin, including for a baby's skin may consist of:
30-90% (w/w) water, more preferably from about 50-80% or from about 60 to 80% or from about 70 to 80% (w/w);
2-35% (w/w) glycerine, more preferably from about 5-25%, or from about 5-20%; 5-33% (w/w) potassium chloride, more preferably from about 5-25%, or from about 8-15%;
0.2-25% (w/w) polysorbate 20 or any other suitable emulsifiers or surfactants known to those skilled in the art, more preferably from about 1 to 10%, or from about 1 to 5%;
0.01-10% (w/w) coco caprylate caprate or any other suitable light emollient oil for skin care known to those skilled in the art;
0.01-20% (w/w) C12-C15 alkyl benzoate;
0.01-2% (w/w) fragrance; and
0.01-3% (w/w) essential oil, including tea tree oil or any other suitable essential oil known to those skilled in the art.

It is to be appreciated that additional anti-microbial and/or anti-inflammatory compounds or compositions including essential oils known to those skilled in the art may be included in the liquid infusion for sensitive skin.

According to a seventh embodiment of the invention, the water-soluble wet wipe is a wet wipe for application of a medical adhesive removing liquid infusion to skin, including medical adhesives used with plasters, stoma bags, adhesive bandages and the like, wherein the medical adhesive removing liquid infusion comprises or consists of:
5-15% (w/w), or 10-15% (w/w);
1-20% (w/w) glycerine, more preferably from about 2-10% or from about 2-5%;
10-50% (w/w) C12-C15 alkyl benzoate, more preferably from about 10-20%;
10-65% (w/w) liquidum paraffinum, more preferably from about 20-50%, or from about 20 to 40%;
5-25% (w/w) hydrogenated castor oil, more preferably from about 5-20% or from about 10-20%;
2-15% (w/w) caprylic/capric triglyceride, more preferably from about 2-10% or from about 5 to 10%;
2-10% (w/w) coco-caprylate, more preferably from about 4-8%;
0.1-3% (w/w) tocopheryl acetate, more preferably from about 0.1-1% or about 0.1-0.5%; 5-15% (w/w) Triticum vulgare (wheat germ oil), more preferably from about 2-10% or from about 2 to 8%;
3-15% (w/w) Jojoba seed oil, more preferably from about 5-15% or from about 8-12%;
0.2-2% (w/w) Lavandula angustifolia (Lavender) oil, more preferably from about 0.5-1.5%, or about 0.8-1.2%;
0.5-3% (w/w) Cymbopogon citratus (Lemongrass) leaf oil, more preferably from about 1.0-2.5% or about 1.5-2.5%;
0.2-3% (w/w) Tea Tree leaf oil, more preferably from about 0.4-0.7%; and
0.2-3% (w/w) Mentha piperita (Peppermint) oil, more preferably from about 0.5-1.5%.

It is to be appreciated that additional anti-microbial and/or anti-inflammatory compounds or compositions including essential oils may be included in the medical adhesive removing liquid infusion.

According to an eighth embodiment of the invention, the water-soluble wet wipe is a wet wipe for application of a cleaning liquid infusion to a lens or screen, including a television, monitor, computer, tablet, or mobile phone screen, or a spectacles lens and the like, wherein the screen or lens cleaning liquid infusion comprises or consists of:
5-15% (w/w), or 10-15% (w/w) water;
1-20% (w/w) glycerine;
55-90% (w/w) alcohol including ethanol or isopropyl alcohol or a mixture of the two, more preferably between about 60-90%, or about 70-90%;
optionally 0-15% (w/w) sodium lauroyl sarcosinate (or a similar surfactant known to those skilled in the art);
optionally 0-18% (w/w) propylene glycol;
optionally 0-1% (w/w) methylchloroisothiazolinone (or a similar preservative known to those skilled in the art); and
optionally 0-1% (w/w) methylisothiazolinone.

More specifically, the liquid infusion of the lens or screen cleaning wipe may consist of:
5-15% (w/w), or 10-15% (w/w) water;
1-20% (w/w) glycerine;
55-90% (w/w) alcohol including ethanol or isopropyl alcohol or a mixture of the two, more preferably between about 60-90%, or about 70-90%;
0.01-15% (w/w) sodium lauroyl sarcosinate (or a similar surfactant known to those skilled in the art);
0.01-18% (w/w) propylene glycol;
0.01-1% (w/w) methylchloroisothiazolinone (or a similar preservative known to those skilled in the art); and 0.01-1% (w/w) methylisothiazolinone.

According to a further aspect of the invention there is provided a method of preparing a water-soluble wet wipe according to the invention comprising a step of infusing a substrate comprising, as the only polymer, a thermally bonded non-woven polyvinyl alcohol fibre of 94% weight/weight (w/w) or more with a liquid infusion comprising or consisting of glycerine and at least 5 or 10% (w/w) water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides such as collagen, and surfactant(s).

In particular the infusion may be performed at a ratio of 1:1.5 up to 1:8.5 (w/w) of the substrate to the liquid infusion respectively. However, more typically, the ratio of substrate to infusion is in the range of 1:2 to 1:8, even more typically 1:2 to 1:6, more typically still 1:2.5 to 1:5 respectively (weight for weight).

The wet wipes of the invention may be processed as a spirally wound roll or as individual wipes. The roll may comprise perforations at regular intervals along the width of the roll for separating individual wipes from the roll.

Individual wipes may be packaged in single sachets or in a resealable flow pack comprising a plurality of layered individual wipes. Alternatively, a spirally wound roll perforated at regular intervals along the width of the roll for separation of individual wipes may be packaged in a resealable canister or in a resealable bucket with a lid.

In one possible embodiment of the invention, the cosmetic wet wipe is shaped to form a mask, sized to fit a person's face and including openings defining the eyes and mouth of the person.

According to a further aspect of the invention there is provided a liquid infusion for infusing a substrate comprising, as the only polymer, a thermally bonded non-woven polyvinyl alcohol fibre of 94% weight/weight (w/w) or more, wherein the liquid infusion comprises or consists of glycerine and at least 5% or at least 10% water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides such as collagen or elastin and the like, and surfactant(s).

According to a further aspect of the invention there is provided a kit of parts comprising (i) a liquid infusion for infusing a substrate comprising, as the only polymer, a thermally bonded non-woven polyvinyl alcohol fibre of 94% (w/w) or more, wherein the liquid infusion comprises or consists of glycerine and at least 5% or at least 10% water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides including collagen and elastin, and surfactant(s), and (ii) a substrate comprising, as the only polymer, a thermally bonded non-woven polyvinyl alcohol fibre of 94% (w/w) or more.

According to a further aspect of the invention there is provided a method of refreshing, sanitizing or disinfecting, repelling insects, removing nail-polish, removing make-up, removing adhesive or cleaning a lens or screen with the application of the liquid infusion by the water-soluble wet wipe of the invention.

There is also provided in a further aspect of the invention the use of a thermally bonded non-woven polyvinyl alcohol fibre of 94% weight/weight (w/w) or more as the only polymer in a substrate for a wet wipe. Typically, the wet wipe comprises a liquid infusion a liquid infusion comprising or consisting of: glycerine and water, and one or more alcohol(s) or one or more metal salt(s), or both one or more alcohol(s) and one or more metal salt(s), and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides such as collagen, elastin and the like, and surfactant(s) according to the first aspect of the invention.

All ranges described herein are modified by the term “about” unless indicated to the contrary. Whilst the invention typically ‘comprises’ the features described herein, the invention may also ‘consist’ or ‘consist essentially of’ the features described herein.

### DETAILED DESCRIPTION OF THE INVENTION

The current invention relates to water soluble wet wipes. The water-soluble wet wipes may in particular be infused with a composition suitable for any one or more of refreshing, sanitizing or disinfecting, repelling insects, removing nail-polish or removing make-up, or cleaning a lens or screen surface.

The following description of the invention is provided as an enabling teaching of the invention, is illustrative of the principles of the invention and is not intended to limit the scope of the invention. It will be understood that changes can be made to the embodiment/s depicted and described, while still attaining beneficial results of the present invention. Furthermore, it will be understood that some benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention.

According to one embodiment, the soluble wet wipe is a refreshing wet wipe for use on the face or body as desired, that consists of a thermally bonded non-woven polyvinyl alcohol fibre of 94% weight/weight (w/w) or more as the only polymer in a substrate, and a liquid infusion consisting of:
water: 10-85% (w/w); and
glycerine: 2-25% (w/w); and
isopropyl alcohol: 0-25% (w/w); and/or
potassium chloride: 0-35% (w/w); and
surfactant(s): 0.5-10% (w/w); and
fragrance: 0.1-5% (w/w); and
optionally henoxyethanol and/or ethylhexylglycerin: 0-1% (w/w); and
optionally one or more solubilizer(s): 0-10% (w/w); and
optionally one or more emulsifier(s): 0-15% (w/w); and
optionally one or more emollient(s): 0-10% (w/w),
   wherein where 0% isopropyl alcohol is included >0% potassium chloride is present, and wherein where 0% potassium chloride is included, >0% isopropyl alcohol is present.

In an alternative embodiment of the invention, the water-soluble wet wipe is a sanitizing or disinfecting wet wipe for use on the body, excluding sensitive areas, or on general surfaces to be sanitised or disinfected as desired, and the liquid infusion comprises or consists of:
water: 5 or 10-25% (w/w);
glycerine: 1-20% (w/w);
isopropyl alcohol: 70-95% (w/w);
optionally hydrogen peroxide: 0-7% (w/w);
peppermint and/or menthol oil(s): 0.5-5% (w/w); and
optionally additional fragrance: 0.05-3% (w/w).

In a third alternative embodiment of the invention, the water-soluble wet wipe is an insect repellent, and optionally also disinfectant or sanitizing wet wipe, for use on the body, particularly uncovered parts of the body that may be exposed to insect bites, and the liquid infusion comprises or consists of:
water: 5 or 10-25% (w/w);
glycerine: 1-25% (w/w);
isopropyl alcohol: 55-95% (w/w);
citronella and/or lemongrass oil: 0.5-25% (w/w); and
surfactant: 0.5-10% (w/w).

The targeted insects are typically mosquitoes, fleas, ticks, flies and the like. In particular the insect is a mosquito.

In a fourth alternative of the invention, the water-soluble wet wipe is a nail-polish remover wet wipe for application of the liquid infusion to the finger or toenails of the body, thereby to remove nail-polish therefrom, wherein the liquid infusion consists of:
water: 5 or 10-20% (w/w);
glycerine: 3-15% (w/w);
alcohol(s): 5-20% (w/w);
ethyl acetate: 0-85% (w/w) and/or acetone: 0-90% (w/w) or a mixture thereof;
essential oil(s): 0.4-10% (w/w); and
additional fragrance(s): 0.2-3% (w/w),
   wherein where 0% acetone is included >0% ethyl acetate is present, and wherein where 0% ethyl acetate is included, >0% acetone is present.

Typically, the alcohol is isopropyl alcohol and the essential oil(s) are a combination of at least sweet almond oil and jojoba oil.

In a fifth alternative of the invention, the water-soluble wet wipe is a cosmetic wet wipe for application of a liquid infusion to the face, including to clean, moisturise or remove make-up therefrom, wherein the liquid infusion comprises or consists of:
water: 10-70% (w/w); and
glycerine: 2-20% (w/w); and
one or more alcohol(s) including isopropyl alcohol: 0-20% (w/w); or
one or more metal salts including potassium chloride or sodium chloride or a combination thereof: 0-30% (w/w); or
both one or more alcohol(s) and one or more metal salt(s); and
DL-Alpha tocopheryl acetate or Vitamin E acetate: 0.1-5% (w/w); and
one or more essential oil(s): 1.8-18% (w/w); and
one or more emulsifier(s) including hydrogenated castor oil and/or polysorbate 20:2-15% (w/w); and
one or more chelating agents including disodium ethylenediaminetetraacetic acid (EDTA): 0.1-0.5% (w/w); and
one or more emollient(s) including isopropyl myristate: 1-7.0% (w/w); and
one or more surfactant(s) including cocamidopropyl betaine: 0.5-5.0 (w/w); and
one or more antioxidants including caprylic/capric triglyceride: 1.0-6.0% (w/w); and
one or more stabilisers and/or preservatives including phenoxyethanol and ethylhexylglycerin or a combination thereof: 0.5-1.0% (w/w); and
optionally mineral oil including paraffinum liquidum: 0-12% (w/w); and
optionally an exfoliant including lactic acid: 0-7% (w/w); and
optionally a hydrating agent including sodium hyaluronate or hyaluronic acid: 0-4% (w/w),
optionally one or more peptide(s) or protein(s) including a hydrolysed peptide or protein such as collagen or elastin: 0-10 (w/w), more preferably 0-2.5% (w/w),
   wherein where 0% alcohol(s) are included >0% metal salt(s) are present, and wherein where 0% metal salt(s) are included, >0% alcohol(s) are present.

In one possible embodiment of the invention, the cosmetic wet wipe is in the shape of a mask, sized to fit a person's face, such that it can be laid onto the surface of the face and pressed down by a user to allow the active infusion to penetrate the skin, followed by removal and disposal of the cosmetic wet wipe mask.

Typically, the alcohol is isopropyl alcohol and the essential oil(s) are a combination of at least tea tree oil (Melaleuca alternifolia): 0.1-2.0% (w/w), lemon oil including Citrus limon oil: 0.2-3.0% (w/w), sweet almond oil (Prunus amagdalis dulcis): 1.0-6.0% (w/w) and Jojoba oil (Simmondsia chinensis): 0.5-7.0% (w/w), but it is to be appreciated that any one or more essential oil(s) known to have moisturising or hydrating properties known to those skilled in the art may be used.

In a sixth alternative of the invention, the water-soluble wet wipe is a wet wipe for application of a liquid infusion to sensitive skin, including for a baby's skin, including to clean or moisturise the skin, wherein the liquid infusion comprises or consists of:
30-90% (w/w) water, more preferably from about 50-80% or from about 60 to 80% or from about 70 to 80% (w/w);
2-35% (w/w) glycerine, more preferably from about 5-25%, or from about 5-20%;
5-33% (w/w) potassium chloride, more preferably from about 5-25%, or from about 8-0.2-25% (w/w) Polysorbate 20 or any other suitable emulsifiers or surfactants known to those skilled in the art, more preferably from about 1 to 10%, or from about 1 to 5%;
optionally 0-10% (w/w) Coco caprylate Caprate or any other suitable light emollient oil for skin care known to those skilled in the art;
optionally 0-20% (w/w) C12-C15 alkyl benzoate;
optionally 0-2% (w/w) fragrance; and
optionally 0-3% (w/w) essential oil, including tea tree oil or any other suitable essential oil known to those skilled in the art.

It is to be appreciated that additional anti-microbial and/or anti-inflammatory compounds or compositions including essential oils known to those skilled in the art may be included in the liquid infusion for sensitive skin.

In a seventh alternative of the invention, the water-soluble wet wipe is a wet wipe for application of a medical adhesive removing liquid infusion to skin, including medical adhesives used with plasters, stoma bags, adhesive bandages and the like, wherein the medical adhesive removing liquid infusion comprises or consists of:
5 or 10-15% (w/w) water, more preferably about 10%; 1-20% (w/w) glycerine, more preferably from about 2-10% or from about 2-5%;
10-50% (w/w) C12-C15 alkyl benzoate, more preferably from about 10-20%;
10-65% (w/w) liquidum paraffinum, more preferably from about 20-50%, or from about 20 to 40%;
5-25% (w/w) hydrogenated castor oil, more preferably from about 5-20% or from about 10-20%;
2-15% (w/w) caprylic/capric triglyceride, more preferably from about 2-10% or from about 5 to 10%;
2-10% (w/w) coco-caprylate, more preferably from about 4-8%;
0.1-3% (w/w) tocopheryl acetate, more preferably from about 0.1-1% or about 0.1-0.5%; 5-15% (w/w) Triticum vulgare (wheat germ oil), more preferably from about 2-10% or from about 2 to 8%;
3-15% (w/w) Simmondsia chinensis (Jojoba) seed oil, more preferably from about 5-15% or from about 8-12%;
0.2-2% (w/w) Lavandula angustifolia (Lavender) oil, more preferably from about 0.5-1.5%, or about 0.8-1.2%;
0.5-3% (w/w) Cymbopogon citratus (Lemongrass) leaf oil, more preferably from about 1.0-2.5% or about 1.5-2.5%;
0.2-3% (w/w) Melaleuca alternifolia (Tea Tree) leaf oil, more preferably from about 0.4-0.7%; and
0.2-3% (w/w) Mentha piperita (Peppermint) oil, more preferably from about 0.5-1.5%.

It is to be appreciated that additional anti-microbial and/or anti-inflammatory compounds or compositions including essential oils may be included in the medical adhesive removing liquid infusion.

In an eighth alternative of the invention, the water-soluble wet wipe is a wet wipe for application of a cleaning liquid infusion to a lens or screen, including a television, monitor, computer, tablet, or mobile phone screen, or a glasses lens and the like, wherein the screen or lens cleaning liquid infusion comprises or consists of:
5 or 10-15% (w/w) water, more preferably about 10%;
1-20% (w/w) glycerine;
55-90% (w/w) alcohol including ethanol or isopropyl alcohol or a mixture of the two, more preferably between about 60-90%, or about 70-90%;
optionally 0-15% (w/w) sodium lauroyl sarcosinate (or a similar surfactant known to those skilled in the art);
optionally 0-18% (w/w) propylene glycol;
optionally 0-1% (w/w) methylchloroisothiazolinone (or a similar preservative known to those skilled in the art); and
optionally 0-1% (w/w) methylisothiazolinone.

The water-soluble wet wipe is produced by infusing the substrate of thermally bonded non-woven polyvinyl alcohol fibre of 94% weight/weight (w/w) or more as the only polymer with the liquid infusion. In particular the infusion process is performed at a ratio of about 1:1.5 up to about 1:8.5 (w/w) or from 1:2.5 to 1:5 (w/w) respectively of the substrate to the liquid infusion.

The wet wipes of the invention can be processed as a spirally wound roll or as individual wipes. The roll would usually have perforations at regular intervals along the width of the roll for separating individual wipes from the roll.

Individual wipes can be packaged in single sachets or in a resealable flow pack that contains a number of layered individual wipes. Alternatively, a spirally wound roll perforated at regular intervals along the width of the roll for separation of individual wipes can be packaged in a resealable canister or in a resealable bucket with a lid.

The cosmetic wet wipe can be shaped into a mask, sized to fit a person's face, with openings defining the person's eyes and mouth.

The invention will be further described by way of the following examples which are not to be construed as limiting in any way the scope of the invention.

### EXAMPLES

### Example 1: Water-Soluble Wet Wipe Characterisation

Water-soluble wet wipe samples were sent to an analytical laboratory for analysis according to ISO 6259-1 & 6259-3 using the test methods set out below. In particular the following characteristics were evaluated: tensile strength, tear resistance and thickness of the samples.

None of the commercial wet wipe samples tested were water-soluble, but the commercial samples included biodegradable or compostable commercial wet wipe samples.

Samples tested included:
A) Dry wipe substrate without the infusion comprised of non-woven polyvinyl alcohol fibre of 94% weight/weight (w/w);
B) Insect repellent infused water-soluble wet wipe consisting of (w/w):
   a. isopropyl alcohol: 80.0%;
   b. water: 7.0%;
   c. glycerine: 3.2%; and
   d. essential oils citronella and lemongrass: 9.8%;
C) Refreshing water-soluble wet wipe consisting of (w/w):
   a. 23% potassium chloride solution: 78% in water;
   b. glycerine: 20%;
   c. isopropyl alcohol: 1.4%; and
   d. fragrance: 0.6%;
D) Sanitising water-soluble wet wipe consisting of (w/w):
   a. isopropyl alcohol: 89%;
   b. water: 7.2%;
   c. glycerine: 3.6%;
   d. peppermint oil: 0.1%;
E) Commercial biodegradable wet-wipe product;
F) Commercial wet-wipe product;
G) Commercial compostable wet-wipe product;
H) Nail polish remover wet wipe consisting of:
   a. water: 5-20% (w/w);
   b. glycerine: 3-15% (w/w);
   c. isopropyl alcohol: 5-20% (w/w);
   d. ethyl acetate: 0-85% (w/w) and/or acetone: 0-90% (w/w) or a mixture thereof;
   e. sweet almond oil and jojoba oil: 0.4-10% (w/w)
   wherein where 0% acetone is included >0% ethyl acetate is present, and wherein where 0% ethyl acetate is included, >0% acetone is present.
I) Cosmetic wipe consisting of (w/w):
   a. water: 10-70% (w/w); and
   b. glycerine: 2-20% (w/w); and
   c. isopropyl alcohol: 0-20% (w/w) or potassium chloride: 0-30% (w/w), or both isopropyl alcohol and potassium chloride; and
   d. DL-alpha tocopheryl acetate or Vitamin E acetate: 0.1-5% (w/w); and
   e. tea tree oil: 0.1-2.0% (w/w), lemon oil including Citrus limon oil: 0.2-3.0% (w/w), sweet almond oil: 1.0-6.0% (w/w) and Jojoba oil: 0.5-7.0% (w/w); and
   f. hydrogenated castor oil and/or polysorbate 20:2-15% (w/w); and
   g. disodium ethylenediaminetetraacetic acid (EDTA): 0.1-0.5% (w/w); and
   h. isopropyl myristate: 1-7.0% (w/w); and
   i. cocamidopropyl betaine: 0.5-5.0 (w/w); and
   j. caprylic/capric triglyceride: 1.0-6.0% (w/w); and
   k. phenoxyethanol and ethylhexylglycerin or a combination thereof: 0.5-1.0% (w/w);
   wherein where 0% alcohol(s) are included >0% metal salt(s) are present, and wherein where 0% metal salt(s) are included, >0% alcohol(s) are present.
J) Medical adhesive remover wipe consisting of (w/w):
   a Glycerine 3%
   b. Water 6 to 10%
   c. C12-C15 alkyl benzoate: 15%;
   d. liquidum paraffinum: 30%;
   e. Hydrogenated Castor Oil: 15%;
   f. Caprylic/Capric Triglyceride: 7%;
   g Coco-Caprylate: 5%;
   h. Tocopheryl Acetate: 0.2%;
   i. Triticum Vulgare (Wheat) Germ Oil: 5%;
   j. Simmondsia Chinensis (Jojoba) Seed Oil: 10%;
   k. Lavandula Angustifolia (Lavender) Oil: 1%;
   l. Cymbopogon Citratus (Lemongrass) Leaf Oil: 2%;
   m. Melaleuca alternifolia (Tea Tree) Leaf Oil: 0.5%; and
   n. Mentha Piperita (Peppermint) Oil: 1%;
K) Sensitive wipe consisting of (w/w):
   a. glycerine: 8.5%;
   b. water: 76.9%;
   c. potassium chloride: 10.3%;
   d. % Polysorbate 20:2.0%;
   e. coco caprylate caprate: 2%
   f. fragrance 0.3%; and
   g. Tea Tree oil 0.5%;
L) Lens cleaning wipe consisting of:
   a. 10-15% (w/w) water;
   b. 1-20% (w/w) glycerine; and
   c. 70-90% (w/w) isopropyl alcohol.

### Tensile Tests-Strength & Elongation

Strength and elongation properties of the tested samples as set out above were measured on a tensile strength testing machine with a moving jaw of 50±5 mm per minute according to ISO 6259-1 & 6259-3. Strips of each wet wipe material tested were standardised to 50 mm strips to obtain results at Newtons/50 mm.

The results are set out in the table below. Warp refers to the parallel vertical threads whereas the weft refers to the parallel horizontal threads that pass back and forth (up and under) through the warp threads.

TABLE 1
Tensile strength and elongation

| | Warp | | Weft | |
|---|---|---|---|---|
| | Maximum | | Maximum | |
| | load | Elongation | load | Elongation |
| | (N/50 | at maximum | (N/50 | at maximum |
| Sample | mm) | load (%) | mm) | load (%) |
| A | 79.1 | 21.9 | 45.3 | 35.2 |
| | 68.8 | 16.7 | 44.4 | 38.4 |
| | 74.9 | 17.4 | 39.4 | 35.0 |
| | 73.1 | 15.7 | 45.2 | 39.8 |
| | 72.9 | 20.0 | 40.8 | 35.1 |
| Average | 73.8 | 18.3 | 43.0 | 36.7 |
| B | 26.9 | 46.6 | 11.6 | 30.5 |
| | 26.6 | 38.8 | 12.1 | 32.4 |
| | 28.6 | 37.5 | 10.1 | 36.7 |
| | 27.1 | 45.1 | 10.7 | 40.3 |
| | 27.4 | 59.5 | 14.2 | 34.6 |
| Average | 27.3 | 45.5 | 11.8 | 34.9 |
| C | 17.3 | 23.8 | 16.2 | 37.0 |
| | 18.7 | 23.3 | 13.5 | 20.4 |
| | 16.8 | 23.3 | 14.3 | 23.9 |
| | 16.5 | 19.2 | 12.5 | 22.6 |
| | 18.6 | 27.3 | 15.0 | 26.7 |
| Average | 17.6 | 23.4 | 14.3 | 26.1 |
| D | 26.6 | 48.7 | 14.7 | 21.7 |
| | 29.0 | 65.3 | 23.4 | 55.8 |
| | 30.6 | 63.0 | 17.5 | 24.5 |
| | 31.9 | 49.9 | 20.8 | 27.4 |
| | 29.4 | 65.2 | 16.0 | 47.4 |
| Average | 29.5 | 58.4 | 18.5 | 35.3 |
| E | 7.5 | 15.5 | 5.1 | 33.3 |
| | 7.2 | 16.2 | 6.1 | 36.4 |
| | 6.6 | 15.4 | 5.7 | 37.9 |
| | 7.1 | 11.5 | 5.6 | 39.3 |
| | 7.3 | 2.7 | 6.1 | 55.4 |
| Average | 7.1 | 12.3 | 5.7 | 40.5 |
| F | 120.2 | 38.5 | 23.5 | 101.1 |
| | 113.8 | 48.0 | 22.8 | 93.0 |
| | 104.7 | 34.8 | 22.5 | 102.1 |
| | 107.3 | 40.1 | 21.7 | 106.2 |
| | 105.5 | 40.0 | 25.3 | 112.1 |
| Average | 110.3 | 40.3 | 23.2 | 102.9 |
| G | 94.6 | 48.1 | 28.1 | 189.6 |
| | 93.2 | 50.3 | 27.9 | 202.1 |
| | 92.2 | 51.2 | 28.0 | 169.3 |
| | 89.1 | 51.6 | 31.5 | 172.2 |
| | 96.9 | 52.1 | 30.5 | 178.7 |
| Average | 93.2 | 50.7 | 29.2 | 182.4 |
| | Warp | | Weft | |
| | Maximum | | Maximum | |
| | load | Elongation | load | Elongation |
| | (N/50 | at maximum | (N/50 | at maximum |
| Sample | mm)* | load (%) | mm)* | load (%) |
| H | 36.7 | 79 | 24.4 | 78 |
| | 40.2 | 107 | 30.9 | 103 |
| | 33.7 | 110 | 37.8 | 106 |
| | 42.2 | 108 | 32.1 | 99 |
| | 39.0 | 87 | 33.1 | 111 |
| | 36.7 | 111 | 29.7 | 108 |
| Average | 38.1 | 100 | 31.3 | 101 |
| I | 37.1 | 59 | 33.4 | 68 |
| | 49.2 | 86 | 35.7 | 77 |
| | 33.0 | 75 | 29.6 | 75 |
| | 40.5 | 99 | 25.4 | 86 |
| | 39.9 | 105 | 33.0 | 88 |
| | 31.4 | 71 | 28.9 | 67 |
| Average | 38.5 | 83 | 30.7 | 77 |
| J | 43.4 | 36 | 32.3 | 45 |
| | 41.4 | 36 | 29.4 | 35 |
| | 47.7 | 38 | 30.5 | 41 |
| | 38.5 | 34 | 29.7 | 39 |
| | 47.3 | 36 | 34.3 | 37 |
| | 47.6 | 36 | 30.9 | 38 |
| Average | 44.3 | 36 | 31.2 | 39 |
| K | 10.6 | 22 | 9.1 | 22 |
| | 13.2 | 32 | 6.6 | 22 |
| | 8.0 | 25 | 8.1 | 22 |
| | 7.5 | 27 | 6.4 | 15 |
| | 7.5 | 25 | 9.5 | 19 |
| | 13.7 | 34 | 9.0 | 21 |
| Average | 10.1 | 28 | 8.1 | 20 |
| L | 12.9 | 22 | 5.1 | 23 |
| | 10.4 | 23 | 9.8 | 58 |
| | 13.9 | 25 | 11.8 | 67 |
| | 8.1 | 19 | 10.3 | 22 |
| | 9.5 | 19 | 11.2 | 26 |
| | 8.5 | 22 | 10.6 | 23 |
| Average | 10.5 | 22 | 9.8 | 36 |

*Measured on 25 mm stripes and multiplied by two.

### Tear Resistance

Tear resistance was conducted according to ASTM D1004-2009, Standard Test Method for Initial Tear Resistance of Plastic Film and Sheeting. A slit was cut into each wet wipe sample, and the strength needed to propagate the tear further was determined by pulling the ends in an Instron tear tester with moving jaws at 50 mm/min. The results are tabulated below.

TABLE 2
Tear resistance

Maximum load (N)

| Sample | Warp | Weft |
|---|---|---|
| A | 27.9 | 16.8 |
| | 26.7 | 18.4 |
| | 26.2 | 17.6 |
| | 23.9 | 18.9 |
| | 23.7 | 16.6 |
| Average | 25.7 | 17.7 |
| B | 7.0 | 4.2 |
| | 6.6 | 4.3 |
| | 8.6 | 3.9 |
| | 6.6 | 3.6 |
| | 8.6 | 4.8 |
| Average | 7.5 | 4.2 |
| C | 5.7 | 4.5 |
| | 4.7 | 4.7 |
| | 4.6 | 4.3 |
| | 5.3 | 4.3 |
| | 4.8 | 3.8 |
| Average | 5.0 | 4.3 |
| D | 5.2 | 3.4 |
| | 5.8 | 6.1 |
| | 5.4 | 5.6 |
| | 5.7 | 3.9 |
| | 5.9 | 4.8 |
| Average | 5.6 | 4.8 |
| E | 2.2 | 1.9 |
| | 2.5 | 1.6 |
| | 2.9 | 1.3 |
| | 3.3 | 1.8 |
| | 3.2 | 1.6 |
| Average | 2.8 | 1.6 |
| F | 43.4 | 9.0 |
| | 50.1 | 8.7 |
| | 46.9 | 11.3 |
| | 43.1 | 10.0 |
| | 44.7 | 8.3 |
| Average | 45.6 | 9.4 |
| G | 39.0 | 9.4 |
| | 35.3 | 11.0 |
| | 32.5 | 10.6 |
| | 33.0 | 8.3 |
| | 36.8 | 9.6 |
| Average | 35.3 | 9.8 |
| H | 16.1 | 11.9 |
| | 12.0 | 16.1 |
| | 17.8 | 16.6 |
| | 25.4 | 14.0 |
| | 26.0 | 12.4 |
| | 22.2 | 14.3 |
| Average | 19.9 | 14.2 |
| I | 18.1 | 17.9 |
| | 20.1 | 11.9 |
| | 20.6 | 14.4 |
| | 18.1 | 9.0 |
| | 17.3 | 18.6 |
| | 19.2 | 13.7 |
| Average | 18.9 | 14.3 |
| J | 19.9 | 12.6 |
| | 16.8 | 15.2 |
| | 16.4 | 12.8 |
| | 18.6 | 19.0 |
| | 21.7 | 17.8 |
| | 19.6 | 13.4 |
| Average | 18.8 | 15.1 |
| K | 5.6 | 3.7 |
| | 5.0 | 2.8 |
| | 5.1 | 4.2 |
| | 6.8 | 3.7 |
| | 5.2 | 3.6 |
| | 6.5 | 3.7 |
| Average | 5.7 | 3.6 |
| L | 7.8 | 6.7 |
| | 8.0 | 4.0 |
| | 9.1 | 6.0 |
| | 8.9 | 5.0 |
| | 6.4 | 4.1 |
| | 4.8 | 3.2 |
| Average | 7.5 | 4.8 |

### Thickness Measurements

The thickness of the infused water-soluble wet wipes (samples B to D) compared with the dry water soluble substrate (A) and commercial products (E to G) was measured with a micrometer capable of reading to one micron. The results are set out in below.

TABLE 3
Thickness of products

| Sample | Thickness (μm) | | | | | | Average |
|---|---|---|---|---|---|---|---|
| A | 304 | 252 | 259 | 233 | 282 | 264 | 256 |
| | 281 | 230 | 289 | 224 | 274 | 260 | |
| | 272 | 242 | 273 | 258 | 190 | 226 | |
| B | 254 | 283 | 180 | 334 | 262 | 284 | 239 |
| | 185 | 236 | 193 | 248 | 247 | 248 | |
| | 227 | 174 | 284 | 225 | 223 | 213 | |
| C | 266 | 355 | 233 | 240 | 194 | 196 | 235 |
| | 243 | 257 | 235 | 244 | 233 | 197 | |
| | 221 | 276 | 255 | 170 | 247 | 170 | |
| D | 258 | 241 | 274 | 229 | 191 | 208 | 235 |
| | 237 | 270 | 308 | 270 | 158 | 190 | |
| | 278 | 243 | 263 | 183 | 178 | 243 | |
| E | 332 | 306 | 293 | 311 | 319 | 310 | 317 |
| | 285 | 344 | 295 | 290 | 316 | 332 | |
| | 306 | 312 | 330 | 325 | 374 | 318 | |
| F | 263 | 280 | 247 | 230 | 245 | 257 | 261 |
| | 270 | 287 | 251 | 248 | 268 | 254 | |
| | 258 | 297 | 251 | 266 | 273 | 245 | |
| G | 257 | 272 | 262 | 298 | 276 | 236 | 267 |
| | 271 | 287 | 295 | 265 | 233 | 250 | |
| | 258 | 263 | 296 | 267 | 243 | 274 | |

| Sample | Thickness (μm) | | | | | Average |
|---|---|---|---|---|---|---|
| H | 252 | 294 | 296 | 347 | 415 | 312 |
| | 386 | 279 | 278 | 354 | 243 | |
| I | 182 | 167 | 190 | 173 | 186 | 172 |
| | 166 | 169 | 162 | 183 | 139 | |
| J | 228 | 171 | 229 | 194 | 229 | 214 |
| | 227 | 200 | 194 | 221 | 250 | |
| K | 209 | 177 | 256 | 193 | 216 | 215 |
| | 214 | 221 | 228 | 196 | 236 | |
| L | 160 | 180 | 252 | 242 | 224 | 207 |
| | 228 | 239 | 161 | 208 | 180 | |

### Results

Based on the tensile strength tests there is a significant increase of an average of 100-300% in the stretchability or elongation strength of the infused water-soluble wet wipes (B to D and H to L) compared with the dry wipe substrate without the infusion (sample A). This means that the infused water-soluble wet wipe material gains significant stretchability compared with the dry wipe substrate, making it effective for use as a body or facial wipe, similar to the commercial test samples which are not water-soluble.

The tear test shows that the ease of tearing the infused water-soluble wet wipes (B to D and H to L) increased by 350-500% which assists physical destruction of the water-soluble wet wipes by a tearing force (as opposed to an elongating force) apart from the chemical deterioration.

The thickness test illustrates that compared with the dry wipe substrate (A), the infused water-soluble wet wipes only thinned a little over 6%.

In addition to the studies performed above, the infused water-soluble wet wipes (B to D and H to L) were found to have a soft and slightly fluffy texture which was pleasant to use on the body or face, similar to the properties of the commercial products E to G. Furthermore, the water-soluble wet wipes (B to D and H to L) were found to be durable when used to wipe the body or face, and were more durable than the commercial biodegradable wet-wipe product E, which came apart in the hands when attempting to use it.

### Conclusion

The infused water-soluble wet wipes (B to D and H to L) were found to be effective for wiping the body or face, having similar or better durability in use and a similar elongation strength to commercial products E to G, as well as a similar pleasing soft and fluffy texture compared with the commercial products, despite being water-soluble, unlike the commercial products tested.

### Example 2: Bactericidal Activity

The bactericidal efficacy of the sanitising water-soluble wet wipe infusion of the invention as described in Example 1 was determined according to SANS 51276 Edition 2 in accordance with the following experimental conditions:

### Test Strains:

Enterococcus hirae ATCC10541
Escherichia coli ATCC10536
Pseudomonas aeruginosa ATCC15442
Staphylococcus aureus ATCC6538
Product Test Concentrations: 0.01%, 1% & 80%

### Appearance of Diluted Test Infusion:

0.01%—Liquid, clear, colourless
1%—Liquid, clear, colourless
80%—Liquid, clear, colourless

| Interfering Substance: | 3.0 g/l Bovine Albumin, Dirty conditions | |
|---|---|---|
| Contact Time: | 1 | minute |
| Test Temperature: | 20° | C. |

| | |
|---|---|
| Rinsing Solution: | Tween 80 (30 g/l) + Saponin |
| | (30 g/l) + Lecithin (3 g/l) |
| Incubation Conditions: | Aerobic incubation: 37° C. ± 1° C. |
| Incubation Media: | Tryptone Soy Agar |

TABLE 4
Bactericidal Activity of water-soluble wet wipe infusion

| | | | | | | Log | |
|---|---|---|---|---|---|---|---|
| | Experimental | Product | Contact | CFU/ML: | CFU/ML: | Reduction | |
| Organism | conditions | Conc. | time | Start | End | (LogR = ≥5) | Evaluation |
| Enterococcus | Obligatory, | 0.01% | 1 | 7.56 | >3.52 | <4.04 | FAIL |
| hirae | Dirty | 1% | minute | 7.56 | >3.52 | <4.04 | FAIL |
| ATCC10541 | Conditions | 80% | | 7.56 | <2.15 | >5.41 | PASS |
| Escherichia coli | Obligatory, | 0.01% | 1 | 7.68 | >3.52 | <4.16 | FAIL |
| ATCC10536 | Dirty | 1% | minute | 7.68 | >3.52 | <4.16 | FAIL |
| | Conditions | 80% | | 7.68 | <2.15 | >5.53 | PASS |
| Pseudomonas | Obligatory, | 0.01% | 1 | 7.61 | >3.52 | <4.09 | FAIL |
| aeruginosa | Dirty | 1% | minute | 7.61 | >3.52 | <4.09 | FAIL |
| ATCC 15442 | Conditions | 80% | | 7.61 | <2.15 | >5.46 | PASS |
| Staphylococcus | Obligatory, | 0.01% | 1 | 7.64 | >3.52 | <4.12 | FAIL |
| aureus | Dirty | 1% | minute | 7.64 | >3.52 | <4.12 | FAIL |
| ATCC6538 | Conditions | 80% | | 7.64 | <2.15 | >5.49 | PASS |

According to SANS 51276, the water-soluble wet wipe infusion, when used at 80% (v/v) concentration has bactericidal activity (logR≥5) under the following test conditions:
Contact time: 1 minute;
Temperature: 20° C.;
Interfering substance: 3.0 g/L Bovine albumin-Dirty conditions;
Test strains: Escherichia coli ATCC10536, Enterococcus hirae ATCC10541, Pseudomonas aeruginosa ATCC15442, and Staphylococcus aureus ATCC6538.

### Example 3: Dissolution Characteristics

The water-soluble wet wipes as described in Example 1 have all been shown to be non-bioaccumulative, non-toxic, and non-hazardous as accredited by the German GefStVO, a regulator of dangerous substances, and are 100% water dissolvable. It has been demonstrated that the time taken for the water-soluble wipes described above to dissolve in an excess of water are:

### Flowing Water:

Room Temperature: <5 seconds;
Cold Water: <30 seconds;
Hot Water: Instantly.

### Still Water:

Room Temperature: <1 minute;
Cold Water: <2:20 minutes;
Hot Water: <2 seconds.

### Example 4: Dissolution Trial

In 500 ml tap water, 3 representative wet wipes (B-D) of the invention were compared with the dry, i.e. non-infused, (A) wipe. Table 5 shows the dissolution properties of each material.

TABLE 5
Dissolution Properties of Sample Wipes

| | Test | Temperature | | Temperature | | Temperature | | Temperature | |
|---|---|---|---|---|---|---|---|---|---|
| | Item | (5° C.) | | (25° C.) | | (30° C.) | | (50° C.) | |
| With stirring | A | <1 | minute | <1 | minute | <20 | seconds | immediate | |
| at 200 rpm | B | <1 | minute | <1 | minute | <45 | seconds | <1 | minute |
| | C | <2 | minutes | <5 | minutes | <1 | minute | <1 | minute |
| | D | <3 | minutes | <5 | minutes | <5 | minutes | <1 | minute |
| Without | A | <5 | minutes | <1 | minute | <1 | minute | <1 | minute |
| stirring | B | <5 | minutes | <5 | minutes | <5 | minutes | <2 | minutes |
| | C | <40 | minutes | <1 | hour | <15 | minutes | <10 | minutes |
| | D | <1.5 | hours | <1.5 | hours | <30 | minutes | <15 | minutes |

There is also provided:
1. A water-soluble wet wipe comprising or consisting of:
   a substrate comprising, as the only polymer, a thermally bonded non-woven polyvinyl alcohol fibre of 94% weight/weight (w/w) of the substrate or more; and
   a liquid infusion comprising or consisting of:
      at least 1% (w/w) glycerine and at least 5% or 10% (w/w) water of the total liquid infusion, and
      one or more alcohol(s) or
      one or more metal salt(s), or
      both one or more alcohol(s) and one or more metal salt(s), and
      optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides including collagen and elastin, and surfactant(s).
2. The water-soluble wet wipe according to item 1, wherein the one or more alcohol(s) are isopropyl alcohol and/or ethyl alcohol, the one or more metal salt(s) are potassium chloride and the one or more fragrance(s) are one or more essential oil(s).
3. The water-soluble wet wipe according to either item 1 or 2 which is a refreshing wet wipe for application of the liquid infusion on the face or body as desired, wherein the liquid infusion comprises or consists of:
   water: 10-85% (w/w); and
   glycerine: 2-25% (w/w); and
   alcohol(s): 0-25% (w/w); and/or
   metal salt(s): 0-35% (w/w); and
   one or more surfactant(s): 0.5-10% (w/w); and
   fragrance: 0.1-5% (w/w); and
   optionally henoxyethanol and/or ethylhexylglycerin: 0-1% (w/w); and
   optionally one or more solubilizer(s): 0-10% (w/w) and;
   optionally one or more emulsifier(s): 0-15% (w/w) and;
   optionally one or more emollient(s): 0-10% (w/w),
   wherein where 0% alcohol(s) are included >0% metal salt(s) are present, and wherein where 0% metal salt(s) are included, >0% alcohol(s) are present.
4. The water-soluble wet wipe according to any one of items 1 to 3 which is for application of the liquid infusion on the face or body of an infant and wherein the alcohol(s) content is between 0-10% (w/w).
5. The water-soluble wet wipe according to either item 1 or 2 which is a sanitizing or disinfecting wet wipe for application of the liquid infusion on the body, excluding sensitive areas including the face or genital area, or on surfaces to be sanitised or disinfected as desired, wherein the liquid infusion comprises or consists of:
   water: 5-25% (w/w), or 10-25% (w/w);
   glycerine: 1-20% (w/w);
   alcohol(s): 70-95% (w/w);
   one or more essential oil(s): 0.5-5% (w/w);
   optionally hydrogen peroxide: 0-7% (w/w); and
   additional fragrance(s): 0.05-3% (w/w).
6. The water-soluble wet wipe according to item 5, wherein the essential oil(s) are any one or more essential oil(s) having antimicrobial, including anti-viral and/or anti-bacterial properties, including peppermint and/or menthol essential oil(s).
7. The water-soluble wet wipe according to either item 1 or 2, which is an insect repellent wipe, and optionally additionally a disinfectant or sanitizing wet wipe, for application of the liquid infusion on the body, including uncovered parts of the body that may be exposed to insect bites, wherein the liquid infusion comprises or consists of:
   water: 6-25% (w/w), or 10-25% (w/w);
   glycerine: 1-25% (w/w);
   alcohol: 55-95% (w/w);
   insect repellent essential oil(s): 0.5-25% (w/w); and
   one or more surfactant(s): 0.5-10% (w/w).
8. The water-soluble wet wipe according to item 7, where in the essential oil(s) are any one or more essential oil(s) having insect repellent properties, including citronella and/or lemongrass.
9. The water-soluble wet wipe according to either item 7 or 8 wherein the application of the liquid infusion to the body is effective for repelling any one or more of mosquitoes, fleas, ticks or flies.
10. The water-soluble wet wipe according to either item 1 or 2 which is a nail-polish remover wet wipe for application of the liquid infusion to the finger or toenails of the body, thereby to remove nail-polish therefrom, wherein the liquid infusion comprises or consists of:
   water: 5-20% (w/w), or 10-20% (w/w);
   glycerine: 3-15% (w/w);
   alcohol(s): 5-20% (w/w);
   ethyl acetate: 0-85% (w/w) and/or acetone: 0-90% (w/w) or a mixture thereof;
   one or more essential oil(s): 0.4-10% (w/w); and
   one or more additional fragrance(s): 0.2-3% (w/w),
   wherein where 0% acetone is included >0% ethyl acetate is present, and wherein where 0% ethyl acetate is included, >0% acetone is present.
11. The water-soluble wet wipe according to item 10, wherein the alcohol is isopropyl alcohol, and the essential oils are a combination of at least sweet almond oil and jojoba oil.
12. The water-soluble wet wipe according to either item 1 or 2 which is a cosmetic wet wipe for application of a liquid infusion to the face, including to clean, moisturise or remove make-up therefrom, wherein the liquid infusion comprises or consists of:
   water: 10-70% (w/w); and
   glycerine: 2-20% (w/w); and
   one or more alcohol(s) including isopropyl alcohol: 0-20% (w/w); or
   one or more metal salts including potassium chloride or sodium chloride or a combination thereof: 0-30% (w/w); or
   both one or more alcohol(s) and one or more metal salt(s); and
   DL-Alpha tocopheryl acetate or Vitamin E acetate: 0.1-5% (w/w); and
   one or more essential oil(s): 1.8-18% (w/w); and
   one or more emulsifier(s) including hydrogenated castor oil and/or polysorbate 20:2-15% (w/w); and
   one or more chelating agents including disodium ethylenediaminetetraacetic acid (EDTA):
   0.1-0.5% (w/w); and
   one or more emollient(s) including isopropyl myristate: 1-7.0% (w/w); and
   one or more surfactant(s) including cocamidopropyl betaine: 0.5-5.0 (w/w); and
   one or more antioxidants including caprylic/capric triglyceride: 1.0-6.0% (w/w); and
   one or more stabilisers and/or preservatives including phenoxyethanol and ethylhexylglycerin or a combination thereof: 0.5-1.0% (w/w); and
   optionally mineral oil including paraffinum liquidum: 0-12% (w/w); and
   optionally an exfoliant including lactic acid: 0-7% (w/w); and
   optionally a hydrating agent including sodium hyaluronate or hyaluronic acid: 0-4% (w/w),
   optionally one or more peptide(s) or protein(s) including a hydrolysed peptide or protein including collagen or elastin: 0-10% (w/w), including 0-2.5% (w/w),
   wherein where 0% alcohol(s) are included >0% metal salt(s) are present, and wherein where 0% metal salt(s) are included, >0% alcohol(s) are present.
13. The water-soluble wet wipe according to item 12, wherein the essential oils are a combination of at least tea tree oil (Melaleuca alternifolia): 0.1-2.0% (w/w), lemon oil including Citrus limon oil: 0.2-3.0% (w/w), sweet almond oil (Prunus amagdalis dulcis): 1.0-6.0% (w/w) and jojoba oil (Simmondsia chinensis): 0.5-7.0% (w/w).
14. The water-soluble wet wipe according to item 12 or 13 which is in the shape of a mask, sized to fit a person's face, such that it can be laid onto the surface of the face and pressed down by a user to allow the active infusion to penetrate the skin, followed by removal and disposal of the cosmetic wet wipe mask.
15. The water-soluble wet wipe according to either item 1 or 2 which is a wet wipe for application of a liquid infusion to sensitive skin, including for a baby's skin, including to clean or moisturise the skin, wherein the liquid infusion comprises or consists of:
   30-90% (w/w) water, including from about 50-80% or from about 60 to 80% or from about 70 to 80% (w/w);
   2-35% (w/w) glycerine, including from about 5-25%, or from about 5-20%;
   5-33% (w/w) potassium chloride, including from about 5-35%, or from about 8-15%;
   0.2-25% (w/w) including from about 1 to 10%, or from about 1 to 5% of one or more emulsifier(s) or surfactant(s) including polysorbate 20;
   optionally 0-10% (w/w) one or more emollient oil(s) including coco caprylate caprate;
   optionally 0-20% (w/w) C12-C15 alkyl benzoate;
   optionally 0-2% (w/w) fragrance; and
   optionally 0-3% (w/w) one or more essential oil(s), including tea tree oil.
16. The water-soluble wet wipe according to item 15, wherein the one or more essential oil(s) are those having anti-inflammatory or antimicrobial properties.
17. The water-soluble wet wipe according to either item 1 or 2 which is a wet wipe for application of a medical adhesive removing liquid infusion to skin, including medical adhesives used with plasters, stoma bags, and adhesive bandages, wherein the medical adhesive removing liquid infusion comprises or consists of:
   5-15% (w/w), or 10-15% (w/w);
   1-20% (w/w), including from about 2-10% or from about 2-5% glycerine;
   10-50% (w/w), including from about 10-20% C12-C15 alkyl benzoate;
   10-65% (w/w), including from about 20-50%, or from about 20 to 40% liquidum paraffinum;
   5-25% (w/w), including from about 5-20% or from about 10-20% hydrogenated castor oil;
   2-15% (w/w), including from about 2-10% or from about 5 to 10% caprylic/capric triglyceride;
   2-10% (w/w) including from about 4-8% coco-caprylate; 0.1-3% (w/w), including from 0.1-1% or about 0.1-0.5% tocopheryl acetate;
   5-15% (w/w), including from about 2-10% or from about 2 to 8% Triticum vulgare (wheat germ oil);
   3-15% (w/w), including from about 5-15% or from about 8-12% Jojoba seed oil;
   0.2-2% (w/w), including from about 0.5-1.5%, or about 0.8-1.2% Lavandula angustifolia (Lavender) oil;
   0.5-3% (w/w), including from about 1.0-2.5% or about 1.5-2.5% Cymbopogon citratus (Lemongrass) leaf oil;
   0.2-3% (w/w), including from about 0.4-0.7% Tea Tree leaf oil; and
   0.2-3% (w/w), including from about 0.5-1.5% Mentha piperita (Peppermint) oil.
18. The water-soluble wet wipe according to item 17, wherein the one or more essential oil(s) are those having anti-inflammatory or antimicrobial properties.
19. The water-soluble wet wipe according to either item 1 or 2 which is a wet wipe for application of a cleaning liquid infusion to a lens or screen, including a television, monitor, computer, tablet, or mobile phone screen, or a spectacles lens, wherein the screen or lens cleaning liquid infusion comprises or consists of:
   5-15% (w/w), or 10-15% (w/w) water;
   1-20% (w/w) glycerine;
   55-90% (w/w), including between about 60-90%, or about 70-90% alcohol, including ethanol or isopropyl alcohol or a mixture of the two;
   optionally 0-15% (w/w) one or more surfactant(s) including sodium lauroyl sarcosinate;
   optionally 0-18% (w/w) propylene glycol;
   optionally 0-1% (w/w) one or more preservative(s) including methylchloroisothiazolinone; and
   optionally 0-1% (w/w) methylisothiazolinone.
20. A method of preparing a water-soluble wet wipe according to any one of items 1 to 13 comprising a step of infusing a substrate comprising, as the only polymer, a thermally bonded non-woven polyvinyl alcohol fibre of 94% (w/w) or more with a liquid infusion comprising or consisting of glycerine and water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides including collagen and elastin, and surfactant(s).
21. The method according to item 20, wherein the infusion is performed at a ratio of about 1:1,5 up to about 1:8,5 (w/w), or from 1:2.5 to 1:5 (w/w) of the substrate to the liquid infusion.
22. The method according to either item 20 or 21, wherein the prepared water-soluble wet wipes of the invention are further processed to form a spirally wound roll optionally including perforations at regular intervals along the width of the roll for separating individual wipes from the roll, or are further processed to form individual wipes.
23. A liquid infusion for infusing a substrate comprising, as the only polymer, a thermally bonded non-woven polyvinyl alcohol fibre of 94% (w/w) or more, wherein the liquid infusion comprises or consists of glycerine and at least 5% or at least 10% water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides including collagen and elastin, and surfactant(s).
24. A method of refreshing the face and/or body, sanitizing or disinfecting the body, repelling insects on the face and/or body, removing nail-polish from the nails and/or removing make-up from the face with the use of the water-soluble wet wipe according to any one of items 1 to 19.
25. A kit of parts comprising (i) a liquid infusion for infusing a substrate comprising, as the only polymer, a thermally bonded non-woven polyvinyl alcohol fibre of 94% (w/w) or more, wherein the liquid infusion comprises or consists of glycerine and at least 5% or at least 10% water; and one or more alcohol(s), and/or one or more metal salt(s); and optionally one or more additives including chelating agent(s), stabiliser(s), emulsifier(s), preservative(s), exfoliating agent(s), hydrating agent(s), anti-oxidants, emollient(s), thickener(s), lubricant(s), acetone or ethyl acetate, essential oil(s), fragrance(s), hydrogen peroxide, one or more proteins or peptides including collagen and elastin, and surfactant(s), and (ii) a substrate comprising, as the only polymer, a thermally bonded non-woven polyvinyl alcohol fibre of 94% (w/w) or more.

1. A water-soluble wet wipe comprising:
(a) a substrate comprising, as the only polymer, a thermally bonded non-woven polyvinyl alcohol fibre of at least 94% weight/weight (w/w) of the substrate or; and
(b) a liquid infusion comprising:
at least 1% (w/w) glycerine;
at least 5% or 10% (w/w) water of the total liquid infusion; and
one or more alcohols, or one or more metal salts, or
both one or more alcohols and one or more metal salts.

2. The water-soluble wet wipe according to claim 1, wherein;
(a) the one or more alcohols are selected from the group consisting of isopropyl alcohol, ethyl alcohol, or both; and
(b) one of the one or more metal salts is potassium chloride.

3. The water-soluble wet wipe according to claim 1, wherein the water-soluble wet wipe is a refreshing wet wipe for application of the liquid infusion on the face or body, wherein:
(a) the liquid infusion comprises:
water: 10-85% (w/w);
glycerine: 2-25% (w/w);
isopropyl alcohol: 0-25% (w/w), potassium chloride: 0-35% (w/w), or both isopropyl alcohol: 0-25% (w/w) and potassium chloride: 0-35% (w/w);
one or more surfactants: 0.5-10% (w/w); and
fragrance: 0.1-5% (w/w); and
(b) where 0% alcohols are included >0% metal salts are present, and where 0% metal salts are included, >0% alcohols are present.

4. The water-soluble wet wipe according to claim 1, wherein the water-soluble wet wipe is for application of the liquid infusion on the face or body of an infant and wherein the alcohol content is between 0-10% (w/w).

5. The water-soluble wet wipe according to claim 1, wherein the water-soluble wet wipe is a sanitizing or disinfecting wet wipe for application of the liquid infusion on the body, excluding sensitive areas selected from the group consisting of the face or genital area, or on surfaces to be sanitised or disinfected, wherein the liquid infusion comprises:
water: 5-25% (w/w);
glycerine: 1-20% (w/w);
one or more alcohols: 70-95% (w/w); and
one or more essential oils: 0.5-5% (w/w).

6. The water-soluble wet wipe according to claim 5, wherein the essential oils are any one or more essential oils having antimicrobial properties.

7. The water-soluble wet wipe according to claim 1, wherein the water-soluble wet wipe is an insect repellent wipe for application of the liquid infusion on parts of a body, wherein the liquid infusion comprises:
water: 6-25% (w/w);
glycerine: 1-25% (w/w);
one or more alcohols: 55-95% (w/w);
one or more insect repellent essential oils: 0.5-25% (w/w); and
one or more surfactants: 0.5-10% (w/w).

8. The water-soluble wet wipe according to claim 7, where in the essential oils are selected from the group consisting of citronella, lemongrass, or both.

9. (canceled)

10. The water-soluble wet wipe according to claim 1, wherein the water-soluble wet wipe is a nail-polish remover wet wipe for application of the liquid infusion to the finger or toenails of a body, thereby to remove nail-polish therefrom, wherein:
(a) the liquid infusion comprises:
water: 5-20% (w/w);
glycerine: 3-15% (w/w);
one or more alcohols: 5-20% (w/w);
ethyl acetate: 0-85% (w/w), acetone: 0-90% (w/w), or a mixture thereof;
one or more essential oils: 0.4-10% (w/w); and
one or more fragrances: 0.2-3% (w/w); and
(b) where 0% acetone is included >0% ethyl acetate is present, and where 0% ethyl acetate is included, >0% acetone is present.

11. The water-soluble wet wipe according to claim 10, wherein the alcohol is isopropyl alcohol, and the essential oils are a combination of at least sweet almond oil and jojoba oil.

12. The water-soluble wet wipe according to claim 1, wherein the water-soluble wet wipe is a cosmetic wet wipe for application of a liquid infusion to a face, wherein:
(a) the liquid infusion comprises:
water: 10-70% (w/w); and
glycerine: 2-20% (w/w); and
one or more alcohols: 0-20% (w/w), one or more metal salts: 0-30% (w/w), or both one or more alcohols and one or more metal salts; and
DL-Alpha tocopheryl acetate or Vitamin E acetate: 0.1-5% (w/w);
one or more essential oils: 1.8-18% (w/w);
one or more emulsifiers: 2-15% (w/w); and
one or more chelating agents: 0.1-0.5% (w/w); and
one or more emollients: 1-7.0% (w/w);
one or more surfactants: 0.5-5.0 (w/w); and
one or more antioxidants: 1.0-6.0% (w/w); and
one or more stabilisers or preservatives or a combination thereof: 0.5-1.0% (w/w); and
(b) where 0% alcohols are included >0% metal salts are present, and where 0% metal salts are included, >0% alcohols are present.

13. The water-soluble wet wipe according to claim 12, wherein the essential oils are a combination of at least tea tree oil (Melaleuca alternifolia): 0.1-2.0% (w/w), lemon oil: 0.2-3.0% (w/w), sweet almond oil (Prunus amagdalis dulcis): 1.0-6.0% (w/w), and jojoba oil (Simmondsia chinensis): 0.5-7.0% (w/w).

14. The water-soluble wet wipe according to claim 12, wherein the water-soluble wet wipe is in the shape of a mask, sized to fit a person's face.

15. The water-soluble wet wipe according to claim 1, wherein the water-soluble wet wipe is a wet wipe for application of a liquid infusion to sensitive skin, wherein the liquid infusion comprises:
water: 30-90% (w/w);
glycerine: 2-35% (w/w);
potassium chloride: 5-33% (w/w); and
emulsifiers, surfactants, or both; 0.2-25% (w/w).

16. The water-soluble wet wipe according to claim 15, wherein the liquid infusion further comprises one or more essential oils having anti-inflammatory or antimicrobial properties.

17. The water-soluble wet wipe according to claim 1, wherein the water-soluble wet wipe is a wet wipe for application of a medical adhesive removing liquid infusion to skin, wherein the medical adhesive removing liquid infusion comprises:
water: 5-15% (w/w);
glycerine: 1-20% (w/w);
benzoate: 10-50% (w/w);
liquidum paraffinum: 10-65% (w/w);
hydrogenated castor oil: 5-25% (w/w);
caprylic/capric triglyceride: 2-15% (w/w);
coco-caprylate: 2-10% (w/w);
tocopheryl acetate: 0.1-3% (w/w);
Triticum vulgare (wheat germ oil): 5-15% (w/w);
Jojoba seed oil: 3-15% (w/w);
Lavandula angustifolia (Lavender) oil: 0.2-2% (w/w);
Cymbopogon citratus (Lemongrass) leaf oil: 0.5-3% (w/w);
Tea Tree leaf oil: 0.2-3% (w/w); and
Mentha piperita (Peppermint) oil: 0.2-3% (w/w).

18. (canceled)

19. (canceled)

20. A method of preparing a water-soluble wet wipe according to claim 1, the method comprising a step of infusing the substrate comprising, as the only polymer, a thermally bonded non-woven polyvinyl alcohol fibre of at least 94% (w/w) of the substrate, with a liquid infusion comprising:
glycerine;
water; and
one or more alcohols, one or more metal salts, or both one or more alcohols and one or more metal salts;
wherein the step of infusing is performed at a ratio of about 1:1,5 up to about 1:8,5 (w/w) of the substrate to the liquid infusion.

21. (canceled)

22. The method according to claim 20, wherein the method further comprises a step of processing the prepared water-soluble wet wipes to form (a) a spirally wound roll having perforations at regular intervals along the width of the roll for separating individual wipes from the roll, or (b) individual wipes.

23. (canceled)

24. A method of refreshing a face or body, sanitizing or disinfecting the body, repelling insects on the face or body, removing nail-polish from nails, or removing make-up from the face, the method comprising a step of using the water-soluble wet wipe according to claim 1 for application of the liquid infusion to the face or body or a portion of the face or body.

25. A kit of parts comprising:
(a) a liquid infusion for infusing a substrate comprising:
glycerine,
at least 5% water; and
one or more alcohols, and/or one or more metal salts, or both one or more alcohols and one or more metal salts; and
(b) a substrate comprising, as the only polymer, a thermally bonded non-woven polyvinyl alcohol fibre of at least 94% (w/w) of the substrate.

26. The water-soluble wet wipe according to claim 1, further comprising one or more additives selected from the group consisting of chelating agents, stabilisers, emulsifiers, preservatives, exfoliating agents, hydrating agents, anti-oxidants, emollients, thickeners, lubricants, acetone or ethyl acetate, essential oils, fragrances, hydrogen peroxide, proteins, peptides, and surfactants.
